SEQUENCE LISTING (3 sequences)

SEQ ID NO: 1 - protein, 6 aa, Artificial Sequence
  Cleavable linkage
RHGSEF

SEQ ID NO: 2 - protein, 5 aa, Artificial Sequence
  Ubiquitin C-terminal sequence
RLRGV

SEQ ID NO: 3 - protein, 6 aa, Artificial Sequence
  Conserved cleavage site sequence
RLLAPI

(19) United States
(12) Patent Application Publication
(10) Pub. No.: US 2003/0211469 A1
(43) Pub. Date: Nov. 13, 2003

(54) Inhibiting hepatitis c virus processing and replication

(76) Inventor: Lloyd Waxman, Kintnersville, PA (US)

Correspondence Address: MERCK AND CO INC, P O BOX 2000, RAHWAY, NJ, 070650907

(21) Appl. No.: 10/333,378

(22) PCT Filed: Jul. 16, 2001
(86) PCT No.: PCT/US01/22335

(51) Int. Cl. C12Q 1/70; A61K 31/33

(52) U.S. Cl. 435/5; 514/183

(57) ABSTRACT

The present invention features methods for inhibiting HCV replication and processing by targeting heat shock protein 90 (HSP90). HSP90 is a cellular chaperone protein that was found to be an essential factor in NS2/3 self-cleavage. HSP90 can be targeted using compounds inhibiting the ability of HSP90 to facilitate NS2/3 cleavage.

### CROSS-REFERENCE TO RELATED APPLICATIONS

The present application claims priority to provisional application U.S. Serial No. 60/219,550, filed Jul. 20, 2000, hereby incorporated by reference herein.

### BACKGROUND OF THE INVENTION

The references cited herein are not admitted to be prior art to the claimed invention.

An estimated 170 million persons are infected with Hepatitis C virus (HCV) worldwide. The infection is usually persistent, and following an asymptomatic period often lasting years, many patients develop chronic liver disease, including cirrhosis and hepatocellular carcinoma.

HCV is a positive strand RNA virus. (Choo, et al., (1989) Science 244, 362-364; and Choo, et al., (1989) Science 2144, 359-362.) The HCV genome encodes a single polyprotein of approximately 3000 amino acids, containing the viral proteins in the order: C-E1-E2-p7-NS2-NS3-NS4A-NS4B-NS5A-NS5B. The NS proteins are thought to be. non-structural and are involved with the enzymatic functions of viral replication and processing of the viral polyprotein. Release of the individual proteins from the polyprotein precursor is mediated by both cellular and viral proteases. (Choo, et al., (1991) P.N.A.S. USA 88, 2451-2455; Takanmizawa, et al., (1991) J. Virol. 65, 1105-1113; Neddermann, et al., (1997) Biol. Chem. 378,469-476; Lohmann, et al., (1996) J. Hepatol. 24, 11-19; and Houghton, et al., (1991) Hepatology 14, 381-388.)

The proteolytic release of mature NS4A, NS4B, NS5A and NS5B is catalyzed by the chymotrypsin-like senine protease contained within the N-terminal domain of NS3, while host cell proteases release C, E1, E2, and p7, and create the N-terminus of NS2 at amino acid 810. (Mizushima, et al., (1994) J. Virol 68, 2731-2734, and Hijikata, et al., (1993) P.N.A.S. USA 90, 10773-10777.)

Cleavage between NS2 and NS3 is catalyzed by the NS2/3 protease. The NS2/3 activity is likely to be essential for replication of HCV in humans. (Kolykhalov, et al., (2000) Journal of virology 74, 2046-51.)

### SUMMARY OF THE INVENTION

The present invention features methods for inhibiting HCV replication and processing by targeting heat shock protein 90 (HSP90). HSP90 is a cellular chaperone protein that was found to be an essential factor in NS2/3 self-cleavage. IHSP90 can be targeted using compounds inhibiting the ability of HSP90 to facilitate NS2/3 cleavage.

Thus, a first aspect of the present invention describes a method of inhibiting HCV replication or processing in a cell infected with HCV comprising the step of providing to the cell an effective amount of an HSP90 inhibitor. The effective amount is an amount sufficient to cause a detectable decrease in HCV replication. Preferably, replication is inhibited at least about 20%, at least about 50%, at least about 75%, or at least about 90%.

Another aspect of the present invention describes a method of inhibiting NS2/3 cleavage in a polypeptide comprising NS2/3 activity. The method involves providing to the polypeptide an effective amount of an HSP90 inhibitor. The effective amount is an amount sufficient to cause a detectable decrease in NS2/3 cleavage. Preferably, NS2/3 cleavage is inhibited at least about 20%, at least about 50%, at least about 75%, or at least about 90%.

A polypeptide “comprising NS2/3 activity” is made up in whole, or in part, by an amino acid region that is derived from a naturally occurring NS2/3 region and possesses NS2/3 autocatalytic activity. An amino acid region derived from a naturally occurring region contains the sequence of a naturally occurring NS2/3 region or is designed based on a naturally occurring region. The NS2/3 autocatalytic activity can be present on a longer length polypeptide.

Another aspect of the present invention features a method of inhibiting HCV replication in a patient infected with HCV. The method involves the step of administering to the patent an effective amount of a HSP90 inhibitor. Patients that can be infected with HCV include humans and chimpanzees. Preferably, the subject is a human.

Another aspect of the present invention describes a method of identifying a NS2/3 processing inhibitor comprising the steps of: (a) measuring the ability of a compound to inhibit HSP90 association to a polypeptide comprising NS2/3 activity; and (b) measuring the ability of the compound to inhibit NS2/3 cleavage.

Another aspect of the present invention describes a method of identifying an HCV replication inhibitor comprising the steps of: (a) measuring the ability of a compound to inhibit HSP90 activity; and (b) measuring the ability of the compound to inhibit HCV replication.

Another aspect of the present invention describes a method of identifying an HCV replication inhibitor comprising the steps of: (a) measuring the ability of a compound to inhibit HSP90 association to a polypeptide comprising NS2/3 activity; and (b) measuring the ability of the compound to inhibit HCV replication.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1. Physical association of NS2/3 with HSP90. (A) Co-immunoprecipitation of NS2/3 with HSP90-specific antibody. [35S] methionine-labeled NS2/3 (810-1615BK), Ubi-849-1207J-BLA, or firefly luciferase synthesized in reticulocyte lysate were immunoprecipitated with anti-HSP90 mAb 3G3 or with the control IgM TEPC-183. (B) Geldanamycin interferes with the association of NS2/3 and HSP90. [³⁵S]-methionine-labeled NS2/3 synthesized in reticulocyte lysate in the absence (lane 1) or presence (lane 4) of 10 HM geldanamycin was immunoprecipitated with the anti-HSP90 mAb 3G3 (lanes 3 and 6) or the control IgM TEPC-183 (lanes 2 and 5).

FIG. 2. HSP90 inhibitors inhibit NS2/3 cleavage in a cell-based assay. Cloned Jurkat cells expressing a fusion protein of NS2/3 in which 3-lactamase activity is the indicator of successful NS2/3 cleavage were treated with either geldanamycin or radicicol. Inhibitor treatment was for 5 hours, followed by addition of cycloheximide to stop protein synthesis (30 minutes) and subsequent addition of the β-lactamase substrate, CCF2. After 2 hours, β-lactamase activity was quantified by fluorescence readings (460 nm/530 nm ratio). Lower 460 nm/530 nm ratios indicate inhibition of NS2/3 cleavage. The geldanamycin (triangles) IC₅₀ is 40 nM and the radicicol (circles) IC₅₀ is 13 nM.

### DETAILED DESCRIPTION OF THE INVENTION

HSP90 is a chaperone protein identified herein as a target for inhibiting HCV replication or processing. Targeting of HSP90 can be achieved using compounds that inhibit the ability of HSP90 to facilitate NS2/3 cleavage.

Chaperone proteins can prevent incorrect interactions within and between non-native proteins and are thought to increase the yield but not rate of folding reactions of many newly synthesized proteins. (Hartl, (1996) Nature 381, 571.) In addition, by mechanisms related to their participation in protein-folding, chaperone proteins help modulate the activities of a variety of signaling proteins, including tyrosine kinases such as p60src (Whitesell, et al., (1994) P.N.A.S. USA 91, 8324), steroid hormone receptors (for review see Pratt, et al., (1997) Endocr. Rev. 18, 306), and nitric oxide synthase (Garcia-Cardena, et al., (1998) Nature 392, 821, Bender, et al., (1999) J. Biol. Chein. 274, 1472).

HSP90 is a chaperone protein involved in regulating the activity of different proteins. (Scheibel, et al., (1998) Biochemical Pharmacology 56, 675-682.) In at least some instances, HSP90 functions as part of a chaperone complex involving partner proteins that assists cellular protein folding and preventing irreversible side- reactions. (Scheibel, et al., (1998) Biochemical Pharmacology 56, 675-682.)

HSP90 Inhibitors

HSP90 inhibitors physically associate with HSP90 and inhibit NS2/3 cleavage and/or HCV replication. The association between HSP90 may be a direct association or may be mediated by other factors such as partner proteins. Preferred HSP90 inhibitors achieve a level of inhibition of at least about 20%, at least about 50%, at least about 75%, or at least about 90%.

HSP90 activity can be inhibited using a variety of compounds that are well known in the art. Such compounds may be used in methods for inhibiting NS2/3 cleavage and/or HCV replication.

The Example section provided below illustrates the ability of compounds well known in the art such as geldanamycin and radicicol to inhibit NS2/3 cleavage. Geldanamycin and radicicol are compounds that have been described as inhibitors of HSP90 activity. (Roe, et al., (1999) J. Med. Chein. 42, 260-266; and Scheibel, et al., (1998) Biochemical Pharmacology 56, 675-682.) Numerous different derivatives of geldanamycin and radicicol are well known in the art. The ability of such derivatives to inhibit NS2/3 cleavage and HCV replication can be determined using standard techniques.

Geldanamycin is an ansamycin antibiotic having anti-tumor activity. (Roe, et al., (1999) J. Med. Chem. 42, 260-266; and Scheibel, et al., (1998) Biochemical Pharmacology 56, 675-682.) Geldanamycin has been described in different references as exerting an anti-tumor drug effect by binding in the ATP- binding site present in the N-terminal domain of HSP90. (Roe, et al., (1999) J. Med. Chem. 42, 260-266; and Scheibel, et al., (1998) Biochemical Pharmacology 56, 675-682, 1998.)

Derivatives of geldanamycin indicated to have anti-tumor activity are described in different references. (See, for example, U.S. Pat. No. 4,261,989 and U.S. Pat. No. 5,932,566, both of which are hereby incorporated by reference herein.) These derivatives provide a class of compounds containing members that are expected to have activity in inhibiting NS2/3 cleavage and/or HCV replication. The ability of a particular compound to inhibit NS2/3 cleavage or HCV replication can be determined using techniques well known in the art.

Radicicol also exerts an anti-tumor drug effect by binding to HSP90 at the N-terminal domain ATP-binding site. (Roe, et al., (1999) J. Med. Chem. 42, 260-266.) Derivatives of radicicol indicated to have anti-tumor activity are described in different references. (See, for example, U.S. Pat. No. 5,597,846 and U.S. Pat. No. 5,977,165, both of which are hereby incorporated by reference herein.) These derivatives provide a class of compounds containing members that are expected to have activity in inhibiting NS2/3 cleavage and/or HCV replication. The ability of a particular compound to inhibit NS2/3 cleavage or HCV replication can be determined using techniques well known in the art.

NS2/3 Cleavage

NS2/3 cleavage is part of HCV polyprotein processing leading to the production of an active NS3 protease. In naturally occurring HCV polypeptide, cleavage between amino acids 1026 and 1027 separating NS2 from NS3 has been found to be dependent upon protein regions of both NS2 and NS3 flanking the cleaved site. (Grakoui, et al., (1993) P.N.A.S. USA 90, 10583-10587; and Komoda, et al., (1994) Gene 145, 221-226.) The cleavage is independent of the catalytic activity of the NS3 protease, as demonstrated with mutational studies. (Grakoui, et al., (1993) P.N.A.S. USA 90, 10583-10587; and Hijikata, et al., (1993) J. Virol. 67, 4665-4675.)

NS2/3 cleavage can be measured in different systems using polypeptides comprising NS2/3 catalytic activity. Examples of such polypeptides include naturally occurring NS2/3 regions having a sufficient amount of NS2/3 for cleavage and derivatives thereof. The NS2/3 cleavage regions can be present along with other polypeptide regions such as those present in an HCV polypeptide or those not present in an HCV polypeptide. Fusion proteins containing NS2/3 cleavage regions can be used to provide for NS2/3 activity and, for example, to provide markers assisting in assaying for NS2/3 activity.

The NS2/3 cleavage reaction has been studied in bacterial, mammalian and insect cells, and following in-vitro translation of the protein. (Grakoui, et al., (1993) P.N.A.S. USA 90, 10583-10587; Selby, et al., (1993) J. Gen. Virol. 74, 1103-1113; Hijikata, et al., (1993) J. Virol. 67, 4665-4675; Santolini, et al., (1995) J. Virol. 69, 7461-7471; D'Souza, et al., (1994) J. Gen. Virol. 75, 3469-3476; and Pieroni, et al., (1997) J. Virol. 71, 6373-6380.) The protein region essential for NS2/3 cleavage activity has been approximately mapped to amino acids 898 to 1207 of the HCV open reading frarne. (Grakoui, et al., (1993) P.N.A.S. USA 90, 10583-10587; Hijikata, et al., (1993) J. Virol. 67, 4665-4675; and Santolini, et al., (1995) J. Virol. 69, 7461-7471.)

The catalytic mechanism of NS2/3 cleavage is unknown but is speculated to be either a metalloprotease or cysteine protease (Wu, et al., (1998) Trends Biochem. Sci. 23, 92-94; and Gorbalenya, et al., (1996) Perspect. Drug Discovery Design, 64-86), and the NS2 N-terminus is believed to be a transmembrane polypeptide (Santolini, et al., (1995) J. Virol. 69, 7461.) Cleavage activity of in-vitro translated NS2/3 is inhibited by EDTA and activity is restored with metal ion re-addition. (Hijikata, et al., (1993) J. Virol. 67, 4665-4675; and Pieroni, et al., (1997) J. Virol. 71, 6373-6380.)

Identifying NS2/3 Processing and HCV Replication Inhibitors

Different assay formats can be employed to identify compounds targeting HSP90 that inhibit NS2/3 processing or HCV replication. An example of a general assay format involves first identifying a compound that interacts with HSP90 and then determining whether such a compound also inhibits NS2/3 cleavage or HCV replication.

Compounds interacting with HSP90 can directly bind to HSP90 or can interact with an HSP90 partner protein such as that present in a chaperone complex. The ability of a compound to interact with HSP90 can be measured in a variety of ways such as carrying out binding assays and physical association assays.

Binding assays measure the ability of a compound to bind to HSP90 or a HSP90 chaperone complex. Different binding assay formats can be performed to measure the ability of a compound to bind to HSP90 or an HSP90 chaperone complex. Examples of different assay formats include competitive and non-competitive. A preferred target for a binding assay is the ATP binding site.

Compounds identified as binding to HSP90 or an HSP90 chaperone complex can be labeled with a detectable moiety and used to evaluate the ability of other compounds to bind to HSP90 or an HSP90 chaperone complex. Suitable detectable moieties include radioisotopes and fluorescent groups. A particular detectable moiety is preferably selected and positioned on a compound such that the moiety will not substantially affect binding to HSP90 or a HSP90 chaperone complex.

Physical association assays measure HSP90 association with a polypeptide comprising NS2/3 fragments acted on by HSP90 or a HSP90 chaperone complex. Techniques for measuring association of HSP90 and NS2/3 include those using binding agents specific for HSP90 or NS2/3. Such binding agents can be used, for example, to indicate the presence of HSP90 and NS2/3 in a gel or column fraction.

An example of specific binding agents are antibodies. Antibodies specific for HSP90 and NS2/3 can be produced using standard immunological techniques. General techniques for producing and using antibodies are described in Ausubel, Current Protocols in Molecular Biology, John Wiley, 1987-1998, and Harlow, et al., Anitibodies, A Laboratory Manual, Cold Spring Harbor Laboratory, 1988.

Compounds identified as interacting with HSP90 or an HSP90 chaperone complex can be further tested for an effect on NS2/3 processing or HCV replication. Inhibition of NS2/3 processing can be assayed for by measuring NS2/3 autocleavage. Inhibition of HCV replication can be assayed for by measuring a change in HCV levels in a subject infected with HCV. Subjects susceptible to HCV infection include chimpanzees. (Major, et al., (1999) Journal of Virology 73, 3317-3325.)

A preferred target for inhibiting NS2/3 processing or HCV replication is the HSP90 ATP binding site. Compounds binding to other sites, for example, those sites affecting the tertiary structure of HSP90, or other HSP90 activities, can be targeted and tested for their ability to inhibit NS2/3 processing or HCV replication.

Assays can be performed using individual compounds or a preparation containing different compounds. A preparation containing different compounds wherein one or more compounds achieves a desired effect such as interacting with HSP90, inhibiting NS2/3 cleavage, or inhibiting HCV replication, can be divided into smaller groups to identify specific compound(s) having a desired effect. In an embodiment of the present invention a test preparation contains at least 10 different compounds in an assay that measures interaction with HSP90, inhibition of NS2/3 cleavage, or inhibition of HCV replication.

### Administration

Compounds targeting HSP90 can be formulated and administered to a patient using the guidance provided herein along with techniques well known in the art. The preferred route of administration ensures that an effective amount of compound reaches the target. Guidelines for pharmaceutical administration in general are provided in, for example, Remington's Pharmaceutical Sciences 18^{th} Edition, Ed. Gennaro, Mack Publishing, 1990, and Modern Pharmaceutics 2^{nd} Edition, Eds. Banker and Rhodes, Marcel Delker, Inc., 1990, both of which are hereby incorporated by reference herein.

Compounds having appropriate functional groups can be prepared as acidic or base salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

Compounds can be administered using different routes including oral, nasal, by injection, transdermal, and transmucosally. Active ingredients to be administered orally as a suspension can be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants.

When administered by nasal aerosol or inhalation, compositions can be prepared according to techniques well known in the art of pharmaceutical formulation. Such compositions may be prepared, for example, as solutions in saline, employing benzyl alcohol or other suitable preservatives, absorption promoters to enhance bioavailability, fluorocarbons, and/or other solubilizing or dispersing agents.

The compounds may also be administered in intravenous (both bolus and infusion), intraperitoneal, subcutaneous, topical with or without occlusion, or intramuscular form, all using forms well known to those of ordinary skill in the pharmaceutical arts. When administered by injection, the injectable solutions or suspensions may be formulated using suitable non-toxic, parenterally-acceptable diluents or solvents, such as Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters or polyethylene glycols, which are solid at ordinary temperatures, but liquidify and/or dissolve in the rectal cavity to release the drug.

Suitable dosing regimens for the therapeutic applications of the present invention are selected taking into factors well known in the art including age, weight, sex and medical condition of the patient; the severity of the condition to be treated; the route of administration; the renal and hepatic function of the patient; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug. The daily dose for a patient is expected to be between 0.01 and 1,000 mg per adult patient per day.

### EXAMPLES

Examples are provided below to further illustrate different features of the present invention. The examples also illustrate useful methodology for practicing the invention. These examples do not limit the claimed invention.

### Example 1

### Production of NS2/3

HCV residues 810-1615 of the BK strain, which includes all of NS2 and most of NS3 (termed 810-1615BK), was produced from the plasmid pCITE 810-1615BK (a gift from Dr. Nicola La Monica) and has been described previously by Pieroni, et al., (1997) J. Virol. 71, 6373. Following plasmid linearization with BLP1, RNA was transcribed with T7 RNA polymerase and purified. Protein translation was in rabbit reticulocyte lysate, 30° C. for 40 minutes using [³⁵S]-methionine as a label. Translation was then blocked by the addition of cycloheximide (250 μM final) and the samples were immediately frozen on dry ice.

Prior to processing experiments, an aliquot of translated NS2/3 was thawed on ice. Processing of 810-1615BK was initiated by addition to room temperature solutions containing Triton X-100 at 1%, as described by Pieroni, et al., (1997) J. Virol. 71, 6373. The distribution of [³⁵S]-labeled proteins on dried gels was determined with a Phosphorimager (Molecular Dynamics). Product bands were quantified and expressed as a proportion of total signal in the gel lane so that variations in gel lane loading were normalized. The product NS2 from 810-1615 BK was used to generate data shown for inhibitor IC₅₀ calculations, due to its migration on gels in a region with less background than the higher molecular weight NS3 fragment.

### Example 2

### Expression of NS2/3 Fusion Protein

The plasmid pM3A, derived from pCDNA (Invitrogen), encodes a fusion protein termed Ubi-849-1207J-BLA that contains ubiquitin at its N-terminus, followed by NS2/3 residues 849-1207 (J strain) linked to bacterial TEM-1 β-lactamase at the C-terminus. The NS3 protease domain in this construct has the inactivating mutation S1165A, which does not affect NS2/3 processing activity. RNA synthesis for this construct is driven by the T7 promoter, and RNA was separately prepared for translation as in Example 1. Upon translation in rabbit reticulocyte lysate, the ubiquitin is immediately cleaved from the protein by cellular ubiquitin hydrolases. (Hochstrasser, (1996) Annual Review of Genetics 30, 405.)

The cleavable linkage (SEQ. ID. NO. 1) is present as ubiquitin↓ArgHisGlySerGluPhe-NS2/3. Translation of this construct inevitably produced some processed NS2 and NS3 products, since NS2/3 processing for the J strain does not require detergent or membranes as does NS2/3 from the BK strain. Translations were limited to 30 minutes for that reason. Quantification of processing at room temperature was by comparison of samples prepared immediately after addition of cycloheximide with later time samples.

### Example 3

### Reconstitution Experiments

NS2/3 (810-1615BK) was synthesized with [³⁵S]-Met-labeling in rabbit reticulocyte lysate as described in Example 1. Lysate containing translated, [³⁵S]-methionine labeled NS2/3 810-1615BK was centrifuged through a spin column containing P-6 polyacrylamide gel (Bio-Rad; exclusion limit 6000 Da) equilibrated in 20 mM Tris-HCl buffer (pH 7.5). Rabbit reticulocyte lysate (Promega) was filtered with Amicon Microcon-10 units to generate a 10 kDa filtrate.

Dilution of the labeled lysate fraction 10-fold into a rabbit reticulocyte lysate filtrate containing solutes less than 10,000 Da (10 kDa filtrate) supported the NS2/3 cleavage to an extent similar to that observed in undiluted lysate as previously described (Darke, et al., (1999) J. Biol. Chem. 274, 34511). The same material diluted into water or buffer failed to process NS2/3.

Commercial rabbit reticulocyte lysate, in addition to cellular components, is supplemented with DTT, KOAc, GTP and creatine phosphate. The filtered lysate was diluted 10-fold into the column buffer or into buffer containing combinations of 1 nmM ATP, 1.5 mM Mg(OAc)₂, 2 mM DTT and 79 mM KOAc. In some samples, the amount of KOAc was varied (20 mM-237 mM) or ATPyS was substituted for ATP. Processing was initiated by the addition of Triton X-100 to 1% and stopped after 30 minutes with an equal volume of SDS sample buffer. The results are shown in Table 1, where activity is expressed as a percent of the amount of processing achieved in the presence of the 10 kDa filtrate control.

TABLE 1
Reconstitution of HCV NS2/3 Processing in Reticulocyte Lysate

| Addition | HCV NS2/3 Processed ^{a} |
|---|---|
| 10 kDa Filtrate | 100 |
| Mg/ATP/DTT | 7.5 |
| Mg/ATPγS/DTT/79 mM KOAc | 24 |
| Mg/ATP/DTT/79 mM KOAc | 68 |
| Mg/ATP/DTT/20 mM KOAc | 20 |
| Mg/ATP/DTT/39.5 mM KOAc | 52 |
| Mg/ATP/DTT/158 mM KOAc | 102 |
| Mg/ATP/DTT/237 mM KOAc | 104 |

^{a} Relative to the amount processed in the presence of 10 kDa filtrate

The components of the 10 ka filtrate that support processing are stable to treatment with heat or trypsin (heat treatment was 100° C. for 3 minutes, and trypsin treatment was 0.1 mg/ml for 2 hours, followed by a 2-fold excess of pancreatic trypsin inhibitor), suggesting a non-proteinaceous nature. Small molecular weight compounds known to be present in rabbit reticulocyte lysate were examined individually and in combination for their ability to support processing.

While no component alone or in pairs completely supported processing, a solution containing ATP and a combination of salts enabled processing at a level comparable to that achieved with the 10 kDa filtrate, as shown in Table 1. Substitution of ATP with ATPyS in this combination caused a loss of processing, suggesting that the ATP contribution requires hydrolysis to ADP (Table 1).

### Example 4

### Inhibition of NS2/3 Processing by Depletino ATP or Using ATP Analogs

To compliment the reconstitution experiments indicating a role for ATP in NS2/3 processing, rabbit reticulocyte lysate containing translated [³⁵S]-labeled NS2/3 (810-1615BK) was depleted of ATP by treatment with glucose plus hexokinase before processing was initiated by the addition of Triton X-100 to 1%. While neither glucose nor hexokinase alone had a significant effect, the combination of the two, which consumes ATP in the phosphorylation of glucose, inhibited processing by 60%. The results are shown in Table 2.

TABLE 2
Effect of ATP Depletion and Non-Hydrolyzable ATP
Analogs on HCV NS2/3 Processing

| Addition | NS2/3 Processed (% of Control) |
|---|---|
| 83 mM Glucose | 101 |
| 0.5 units Hexokinase | 88 |
| Glucose + Hexokinase | 38 |
| 5 mM Mg/ATPγS | 24 |
| 5 mM Mg/AMP-PNP | 40 |
| [³⁵S]-methionine labeled NS2/3 810-1615BK was synthesized in rabbit reticulocyte lysate and 5 μl aliquots were directly combined with 1 μl 500 mM glucose, 0.5 unit yeast hexokinase, or glucose plus hexokinase, and incubated for 30 minutes at room temperature. Similarly, lysate containing NS2/3 was incubated with Mg/ATPγS or Mg/AMP-PNP, for final concentrations of the nucleoside analogs of 5 mM. A stock solution of Triton | |
| # X-100 at 10% (w/v) was used to initiate autoprocessing of the 810-1615BK NS2/3. Following addition of SDS sample buffer, samples were heated to 100° C. for 5 minutes and proteins separated on SDS/14% polyacrylamide gels. Quantification of products was by phosphorimaging of the dried gels. | |

Alternatively, inhibition was observed with the addition of either ATPγS or AMP-PNP, non-hydrolyzable analogs of ATP (Table 2). Titration of the inhibition yielded IC₅₀ values of 2 mM and 4 mM for ATPγS or AMP-PNP, respectively, with inhibition at the maximum concentration tested (5 mM) of 77% and 60%, respectively (residual ATP is also present in these reactions, at a concentration of approximately 1 mM, so that complete inhibition is not expected). Inhibition by ATPγS was also observed with NS2/3 from the J-strain of HCV, expressed as a fusion protein consisting of ubiquitin-NS2/3-β-lactamase (Ubi-849-1207J-BLA).

### Example 5

### Inhibition of NS2/3 Activity

The involvement of ATP is consistent with the participation of ATP-dependent cellular chaperones at a stage in the processing. Geldanamycin and herbimycin A, two related benzoquinone ansamycins, and radicicol, a macrocyclic antibiotic, are examples of compounds that specifically inhibit HSP90 by binding at the ATP site. (Roe, et al., (1999) J. Med. Chem. 42, 260.)

Inhibition of NS2/3 processing was observed with geldanamycin, herbimycin A, and radicicol when added to in vitro synthesized precursor 810-1615BK (up to 50%). Somewhat greater inhibition of processing was observed, in a dose-dependent manner, if compounds were included during the synthesis phase of the experiment as well as in NS2/3 processing (Table 3). The compounds had no effect on overall efficiency of protein synthesis and similar potencies of inhibition were observed using the NS2/3 fusion protein Ubi-849-1207J-BLA.

TABLE 3
Effect of Inhibitors of HSP90 on HCV NS2/3 Processing

| Addition | NS2/3 Processed (% of Control) |
|---|---|
| 1 μM Geldanamycin | 53 |
| 10 μM Geldanamycin | 33 |
| 1 μM Herbimycin | 38 |
| 10 μM Herbimycin | 22 |
| 1 μM Radicicol | 58 |
| 10 μM Radicicol | 46 |

[³⁵S]-methionine labeled NS2/3 8101615BK was synthesized in reticulocyte lysate in the presence of either 1 or 10 μM geldanamycin, herbimycin A, or radicicol. After blocking further synthesis with cycloheximide, an aliquot was removed and processing was initiated with the addition of Triton X-100 to 1%. After 30 minutes the reaction was terminated with SDS sample buffer and heated to 95° C.

NS2/3 processing reactions were performed with 810-1615BK. The inhibitory effects of geldanamycin and radicicol were titrated using techniques described previously for peptide inhibition titrations. (Darke, et al., (1999) J. Biol. Chem. 274, 34511.) Inhibitors were dissolved in DMSO and protected from light. Dilutions were in DMSO, such that the final concentration of DMSO was 2% for in vitro experiments and 1% for cell-based assays. Titration of geldanamycin and radicicol yielded EC₅₀'s in the low micromolar range (Table 4), similar to what has been observed in analogous in vitro studies of other proteins acted on by HSP90. (Hu, et al., (1996) P.N.A.S. USA 93, 1060; Thulasiraman, et al., (1996) Biochemistry 35, 13443 (1996), Schneider, et al., (1996) P.N.A.S. USA 93, 14536.)

TABLE 4
Inhibition of NS2/3 by HSP90 Inhibitors

| | | Maximum | |
|---|---|---|---|
| | | Inhibition | EC₅₀ |
| Compound Name | Structure | (%) | (μM) |
| Geldanamycin | 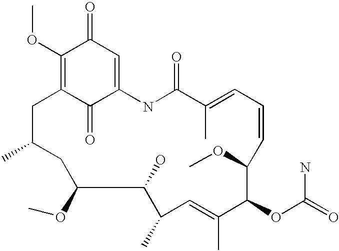 | 91 | 1.8 ±0.5 |
| Radicicol | 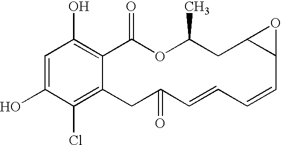 | 51 | 0.22 ± 0.12 |

The IC₅₀ values were determined by first expressing the product level found as a fraction of the no-inhibitor control product level, then fitting the equation

$$Fractional Activity = a + \frac{b}{{( 1 + x / c )}^{d}}$$

to the data, where a is the minimal level of fractional activity (tending to 0), a+b is the maximal level (tending to 1), x is the concentration of inhibitor, c is the IC₅₀ and d is a slope coefficient. For both inhibitors, inhibition leveled out at the maximum extent indicated in the table, so that an effective concentration (EC₅₀) is used to describe the relative potency. Values given are the average of two determinations.

### Example 6

### Physical Association Between NS2/3 and HSP90

Evidence for a physical association of iii vitro translated NS2/3 with HSP90 was obtained by immunoprecipitation. The monoclonal IgM antibodies, 3G3 (anti-HSP90, Affinity Bioreagents) and TEPC-183 (control, Sigma), have been previously described for use in immunoprecipitation of HSP90. (McGuire, et al., (1994) Molecular and Cellular Biology 14, 2438.) Luciferase RNA was obtained from Promega. Immunoaffinity beads were prepared by binding the primary antibody to a solid support by means of a bridging antibody.

Protein G-agarose (Boehringer) was used to immunobilize goat anti-mouse immunoglobulin M (IgM) (5 mg/ml gel) overnight at 4° C. The monoclonal anti-HSP90 antibody 3G3 or an equal concentration of control mouse IgM antibody TEPC-183 was then combined with the immobilized anti-mouse IgM. To immunoprecipitate HSP90 and any associated proteins, lysate containing translated [35 S]-labeled NS2/3 was incubated with the beads essentially as described. (McGuire, et al., (1994) Molecular and Cellular Biology 14, 2438.) Following binding for 2 hours at 4° C., the beads were washed, suspended in SDS sample buffer, and heated to 95° C. Immunoprecipitates were resolved on SDS/14% polyacrylamide gels.

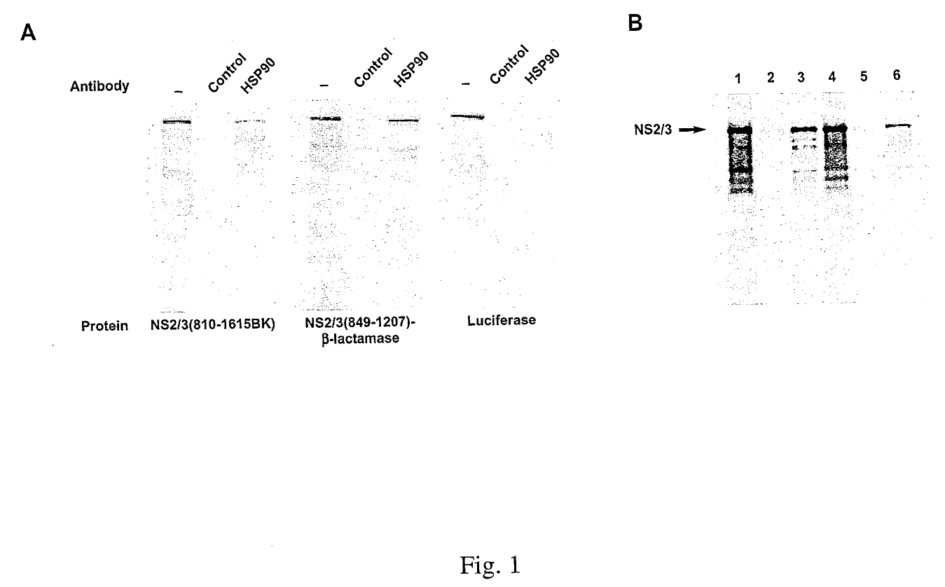

Immunoprecipitation of HSP90 with a monoclonal IgM antibody co-immunoprecipitated NS2/3 derived from either BK or J strains, 810-1615BK and Ubi-849-1207J-BLA, as shown in FIG. 1. A control IgM antibody, TEPC-183 immunoprecipitated only minimal amounts of the protein of interest. Association with HSP90 was not observed, however, with a control protein, translated firefly luciferase (FIG. 1). The results indicate that de novo synthesized NS2/3 forms a stable complex with HSP90 in solution.

### Example 7

### Inhibition of HSP90 Association With NS2/3

The ability of geldanamycin to interfere with the immunoprecipitation of HSP90 with NS2/3 was examined. [³⁵S]-labeled NS2/3 was synthesized in reticulocyte lysate in the absence or presence of 10 μM geldanamycin. In the presence of the geldanamycin the amount of NS2/3 co-immunoprecipitated with the anti- HSP90 mAb was decreased by 60% (FIG. 1B). Thus, some inhibition of NS2/3 processing by geldanamycin may be attributable to the prevention of HSP90 association with NS2/3 during or immediately following translation.

### Example 8

### Cell-Based Inhibition of NS2/3 Cleavage

Validation of the concept that HSP90 is essential for NS2/3 processing in living cells was obtained by treating cells expressing NS2/3 with HSP90 inhibitors. Through the use of a neomycin-selectable transfection vector, stable expression of NS2/3 in Jurkat cells was obtained. The plasmid pUbBla3X-NS2/3-3A conferring neomycin (G418) resistance, expresses a fusion of the NS2/3 protein with 3 ubiquitin domains appended to the N-terminus and β-lactamase at the C-terminus. The complete uncleaved protein has an in vivo half-life estimated to be 5-10 minutes.

The plasmid pUbBla3X NS2/3-3A was transfected into Jurlcat cells. The CMV promoter-driven ORF of the plasmid encodes a 91 kDa protein, ubiquitin-ubiquitin-ubiquitin-NS2/3-β-lactamase, with the C-termini of the 3 ubiquitin domains rendered non-cleaveable to ubiquitin-C-terminal hydrolases (ubiquitin C-terminal sequence ArgLeuArgGlyVal, SEQ. ID. NO. 2). The NS2/3 region includes HCV residues 849-1207. The β-lactamase domain is TEM-1 from E. coli. Expression of the P-lactamase moiety is readily detected with the fluorogenic, cell-penneant substrate, CCF2. (Zlokarnik, et al., (1998) Scienice 279, 84.) Transfectants were sorted by FACS using treatment with CCF2 to indicate β-lactamase expression (Zlokarnik, et al., (1998) Science 279, 84), and individual clones were grown under G418 selection. The full fusion protein expressed is highly unstable to ubiquitin-directed proteosomal degradation due to its ubiquitin N-terminal tag, while the C-terminal product of NS2/3 cleavage, NS3-β-lactamase, is stable for hours. Thus, build-up in the cells of O-lactamase activity, as indicated by CCF2 hydrolysis (high 460 nm1530 nm ratio) is indicative of successful NS2/3 cleavage, and suppression of P-lactamase activity indicates NS2/3 inhibition.

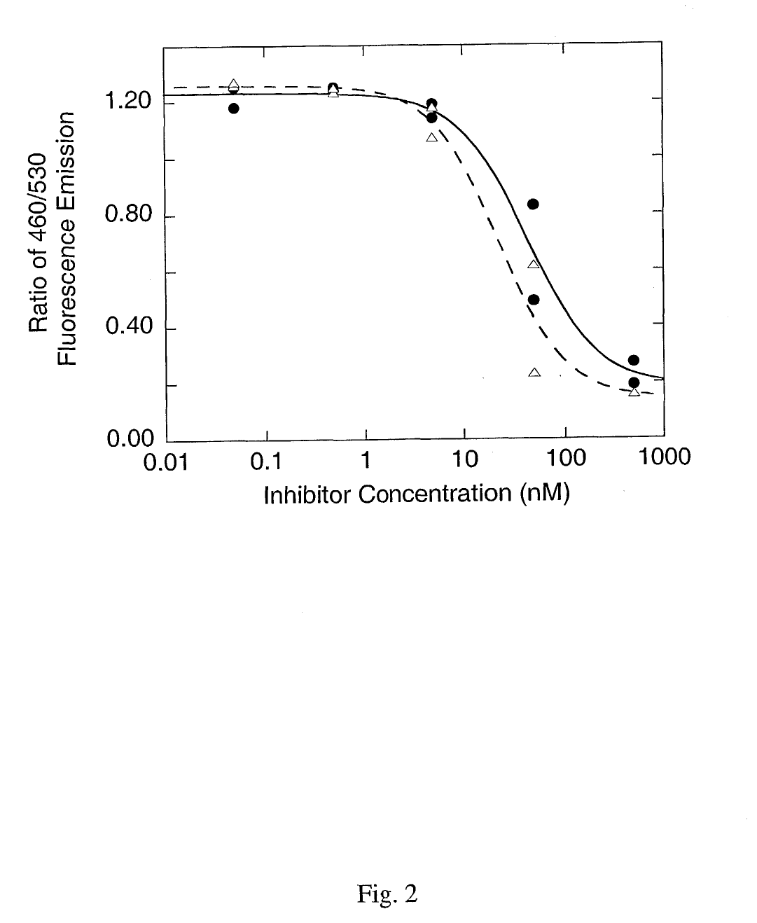

Within this context, the NS2/3 mutation C993A, which is incapable of processing, reduces β-lactamase activity of the cells approximately 8-fold. The protein region essential for NS2/3 cleavage activity has been approximately mapped to amino acids 898 to 1207 of the HCV open reading frame. The conserved cleavage site sequence is Arg↓LeuLAlaProIle (SEQ. ID. NO. 3). Cys993 and His952 have been identified as essential residues. (See, Grakoui, et al., (1993) P.N.A.S. USA 90, 10583, Selby, et al., (1993) J. Gen. Virol. 74, 1103, fijikata, et al., (1993) J. Virol. 67, 4665, Santolini, et al., (1995) J. Viol. 69, 7461, D'Souza, et al., (1994) J. Gen. Virol. 75, 3469, and Pieroni, et al., (1997) J. Virol. 71, 6373.) NS2/3 self-cleavage separates the destabilizing ubiquitin degradation signal at the N-terminus from the NS3-O-lactamase C-terminal product, thus stabilizing the β-lactamase activity within the cell. Using this system the inhibitory potency of HSP90 inhibitors toward NS2/3 processing in mammalian cells was measured, as shown in FIG. 2. Geldanamycin and radicicol are potent inhibitors of NS2/3 cleavage in this context, with IC₅₀ values of 40 nM and 13 nM, respectively. In addition, inhibition is nearly complete at the highest concentrations tested (FIG. 2). The results are comparable to what others have noted for other HSP90 activities, in that the concentration of geldanamycin required to inhibit HSP90 activity in cells is much lower than required in vitro. (Hu, et al., (1996) P.N.A.S. USA 93, 1060, Holt, et al., (1999) Genes Dev. 13, 817.)

Geldanamycin specifically interacts with HSP90 in cells, as demonstrated by affinity labeling (Chavany, et al., (1996) J. Biol. Chem. 271, 4974), and affinity chromatography (Schneider, et al., (1996) P.N.A.S. USA 93, 14536, Whitesell, et al., (1994) P.N.A.S. USA 91, 8324, and Schulte, et al. (1998), Cell Stress and Chaperones 3, 100). Thus, the observed inhibition for NS2/3 cleavage is HSP90-mediated.

Other embodiments are within the following claims. While several embodiments have been shown and described, various modifications may be made without departing from the spirit and scope of the present invention.

What is claimed is:

1. A method of inhibiting Hepatitis C virus (HCV) replication or processing in a cell infected with HCV comprising the step of providing to said cell an effective amount of an HSP90 inhibitor.

2. The method of claim 1, wherein said HSP90 inhibitor inhibits ATP binding to HSP90.

3. The method of claim 1, wherein said HSP90 inhibitor is geldanamycin.

4. The method of claim 1, wherein said HSP90 inhibitor is herbimycin A.

5. The method of claim 1, wherein said HSP90 inhibitor is radicicol.

6. The method of any one of claims 1-5, wherein said method is performed in vitro.

7. The method of claim 1, wherein HCV processing is inhibited.

8. The method of claim 1, wherein HCV replication is inhibited.

9. A method of inhibiting NS2/3 cleavage comprising the step of providing to a polypeptide comprising NS2/3 activity an effective amount of an HSP90 inhibitor.

10. The method of claim 9, wherein said HSP90 inhibitor is geldanamycin, herbimycin A, or radiciciol.

11. The method of claim 9, wherein said HSP90 inhibitor inhibits ATP binding to HSP90.

12. A method of inhibiting HCV replication in a patient comprising the step of administering to said patent an effective amount of a HSP90 inhibitor.

13. The method of claim 12, wherein said HSP90 inhibitor inhibits ATP binding to HSP90.

14. The method of claim 12, wherein said HSP90 inhibitor is geldanamycin, radicicol, or herbimycin A.

15. A method of identifying a NS2/3 processing inhibitor comprising the steps of:
a) measuring the ability of a compound to inhibit HSP90 association to a polypeptide comprising NS2/3 activity; and
b) measuring the ability of said compound to inhibit NS2/3 cleavage.

16. A method of identifying an HCV replication inhibitor comprising the steps of:
a) measuring the ability of a compound to inhibit HSP90 activity; and
b) measuring the ability of said compound to inhibit HCV replication.

17. The method of claim 16, wherein said step (a) measures the ability said compound to inhibit HSP90 to binding ATP.

18. A method of identifying an HCV- replication inhibitor comprising the steps of:
a) measuring the ability of a compound to inhibit HSP90 association to a polypeptide comprising NS2/3 activity; and
b) measuring the ability of said compound to inhibit HCV replication.
